(19) United States
(12) Patent Application Publication
(10) Pub. No.: US 2026/0294521 A1
(43) Pub. Date: Oct. 1, 2026

(54) ELECTROSURGICAL DEVICE

(71) Applicant: KYLIX S.R.L., Napoli (NA) (IT)

(72) Inventor: Giulia DI CAPUA, Napoli (NA) (IT)

(21) Appl. No.: 19/479,386

(22) PCT Filed: Oct. 24, 2024
(86) PCT No.: PCT/IB2024/060460

§ 371 Date: Oct. 28, 2025

(30) Foreign Application Priority Data
Oct. 25, 2023 (IT) 102023000022395

(51) Int. Cl. A61B 18/14; A61B 18/00; A61B 18/12

(52) U.S. Cl. CPC A61B 18/1482; A61B 18/1206; A61B 2018/00595; A61B 2018/00601; A61B 2018/1472

(57) ABSTRACT

The electrosurgical device (1) comprises:—at least one substantially hollow main body (2) of elongated conformation and provided with a proximal end (3), connectable to a current generator (5) and to feeding means (6) of an electro-conductive fluid (7), and with a distal end (4) provided with at least one active electrode (8) and with at least one neutral electrode (9) adapted to contact the body of a patient; and —at least one tubular duct (10) passing through the main body (2) and provided with at least one feeding hole (11) of the fluid (7) and with at least one dispensing hole (12) of the fluid (7); a gripping portion (13) associated with the main body (2); wherein gripping portion (13) comprises coupling means (17) graspable by an operator or associable with at least one gripping device (18) of a robotic movement device (19) of the electrosurgical device (1).

### TECHNICAL FIELD

The present invention relates to an electrosurgical device.

### BACKGROUND ART

To date, the use of electrosurgical devices is widely used in a variety of medical fields, including e.g. ophthalmology, gynecology, cardiology, urology, neurology and oncology.

The electrosurgical devices of known type are provided with a substantially hollow main body, of substantially elongated conformation and provided with a first extremal portion, which can be grasped by an operator, and with a second extremal portion, intended to contact a patient's body.

In detail, the electrosurgical devices of known type are provided with an active electrode and with a neutral electrode, associated protruding with the second extremal portion.

The active electrode is electrically chargeable and is positionable in contact with the patient's body; the neutral electrode is configured to collect the electric current coming out of the active electrode by generating an electric discharge that strikes the tissue of the patient to be treated.

Due to the thermal effect induced by the electrical discharge thus generated, it is possible to achieve the transformation of the cells of the affected tissue, leading, for example, to their cutting or cauterization.

In this regard, the first extremal portion is connectable to an electric current generator configured to electrically charge the active electrode.

The electrosurgical devices of known type are conveniently provided with means of turning on/off the active electrode which are configured to allow/prevent the passage of the electric current from the current generator to the active electrode. In addition, the electrosurgical devices of known type comprise a tubular duct arranged within the main body and configured to dispense an electro-conductive fluid between the active electrode and the neutral electrode in order to facilitate the passage of electric current between these electrodes.

In detail, the tubular duct is provided with an inlet hole, made on the first extreme portion and connectable to an electro-conductive fluid feeding unit, and with at least one outlet hole, made on the second extreme portion and in the proximity of the electrodes.

The electrosurgical devices of known type also comprise means of controlling the dispensing of the electro-conductive fluid configured to adjust the flow of the electro-conductive fluid from the feeding unit to the tubular duct.

The electrosurgical devices of known type do however have some drawbacks that are mainly related to the difficulties encountered when performing medical treatment operations on tissues that require high precision.

In particular, the electrosurgical devices of known type cannot be easily handled in confined spaces and cannot always be employed to perform fine medical-surgical treatments.

### DESCRIPTION OF THE INVENTION

The main aim of the present invention is to devise an electrosurgical device which allows performing medical treatment operations on a patient's tissues that require high precision.

A further object of the present invention is to devise an electrosurgical device which can also be easily used during very delicate and fine surgery operations and/or to be performed in confined spaces.

Another object of the present invention is to devise an electrosurgical device which allows the aforementioned drawbacks of the prior art to be overcome within the framework of a simple, rational, easy and effective to use as well as affordable solution.

The aforementioned objects are achieved by this electrosurgical device having the characteristics of claim 1.

### BRIEF DESCRIPTION OF THE DRAWINGS

Other characteristics and advantages of the present invention will become more apparent from the description of a preferred, but not exclusive, embodiment of an electrosurgical device, illustrated by way of an indicative, yet non-limiting example, in the accompanying tables of drawings in which:

FIG. 1 is an axonometric view of an electrosurgical device according to the invention;

FIG. 2 is a top view of the electrosurgical device according to the invention;

FIG. 3 is a rear view of the electrosurgical device according to the invention;

FIG. 4 is a front view of the electrosurgical device according to the invention;

FIG. 5 is an exploded view of the electrosurgical device according to the invention;

FIG. 6 is an axonometric view of a kit for the electrosurgical treatment of a patient according to the invention.

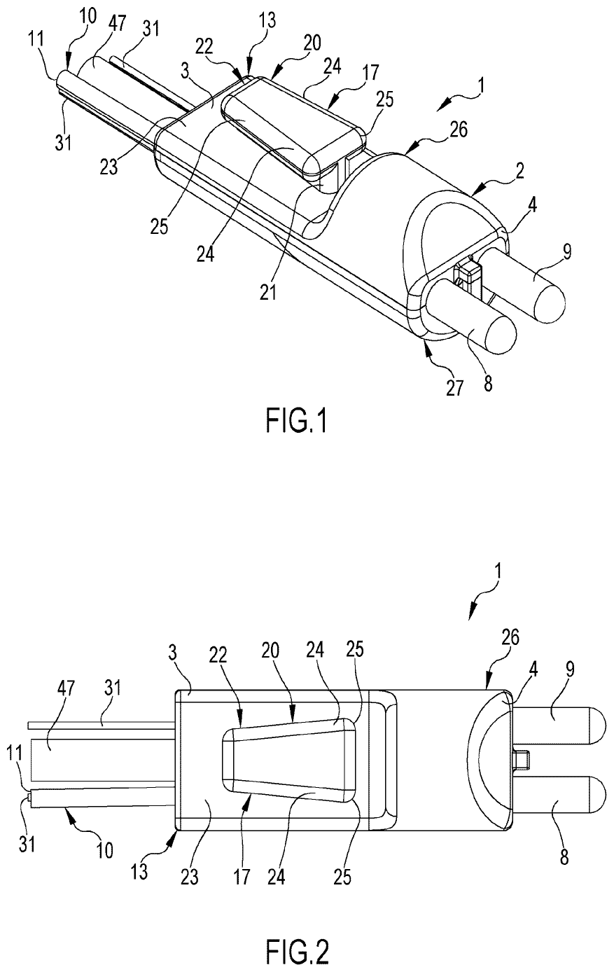

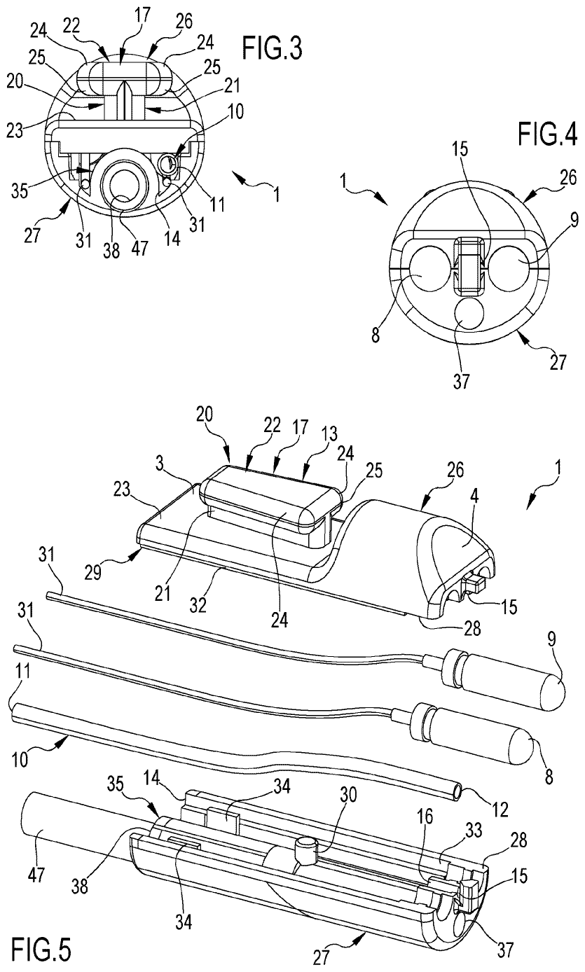

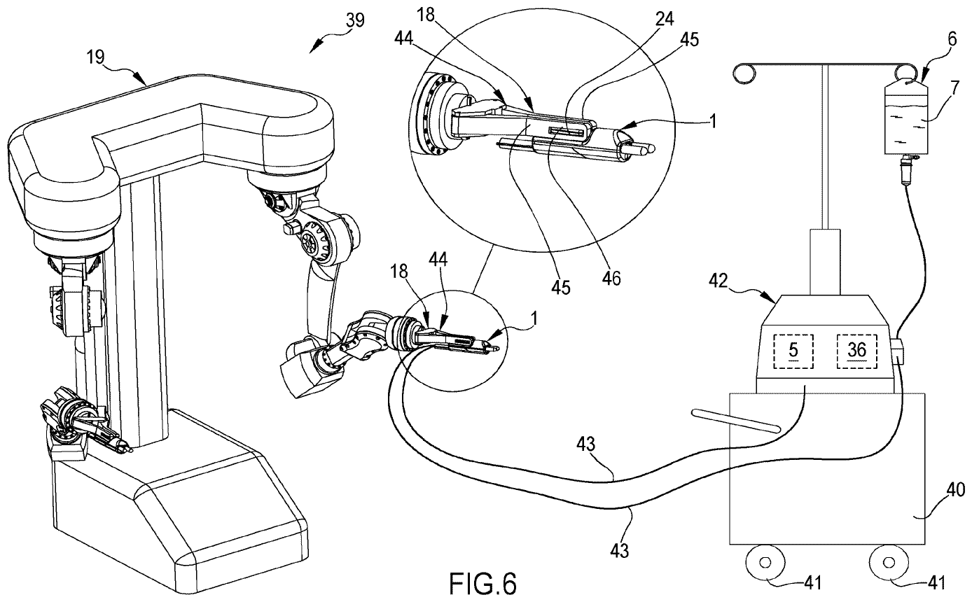

### EMBODIMENTS OF THE INVENTION

With particular reference to these figures, reference numeral 1 globally denotes an electrosurgical device.

The electrosurgical device 1 comprises:
at least one substantially hollow main body 2 of elongated conformation and provided with a proximal end 3, connectable to a current generator 5 and to feeding means 6 of an electro-conductive fluid 7, and with a distal end 4 provided with at least one active electrode 8 and with at least one neutral electrode 9 adapted to contact the body of a patient; and
at least one tubular duct 10 passing through the main body 2 and provided with at least one feeding hole 11 of the fluid 7 and with at least one dispensing hole 12 of the fluid 7;
a gripping portion 13 associated with the main body 2.

It should be emphasized that in the context of this disclosure the terms “distal” and “proximal” and those derivable from them must be considered in relation to the position taken with respect to the current generator 5 and to the feeding means 6.

Preferably, the current generator 5 is configured to generate a radio-frequency current and is between 20 watts and 200 watts in power.

Preferably, the fluid 7 is of the type of a saline solution.

Conveniently, the electrosurgical device comprises means of turning on/off the active electrode 8 which are configured to allow/prevent the passage of electric current from the current generator 5 to the active electrode 8.

Advantageously, the electrosurgical device comprises adjustment means configured to adjust the flow rate of the fluid 7 toward the tubular duct 10. The on/off and the adjustment means are not shown in the figures for pure representational simplicity but are of a type known to the technician in the field. With particular reference to the preferred embodiment shown in the figures, the active electrode 8 and the neutral electrode 9 have a substantially domed conformation.

The electrodes 8, 9 extend cantilevered from the distal end 4.

Each electrode 8, 9 is connectable to the current generator 5 by means of electrical connection elements 31 that are allocated, at least partly, within the main body 2. With particular reference to the preferred embodiment shown in the figures, the tubular duct 10 is allocated within the main body 2 so that the feeding hole 11 protrudes from the proximal end 3.

In this way, the feeding hole 11 can be connected directly to the feeding means 6.

Conveniently, the main body 2 comprises, at the proximal end 3, a through opening 14 from which the tubular duct 10 protrudes.

The tubular duct 10 is arranged within the main body 2 so that the dispensing hole 12 is allocated at the distal end 4.

In more detail, the main body 2 comprises, at the distal end 4, at least one outlet hole 15 connected to the dispensing hole 12 in a fluid-operated manner and adapted to allow the fluid 7 moved by the tubular duct 10 to flow outwards.

The dispensing hole 12 is arranged opposite the through opening 14 along the axis of longitudinal development of the main body 2.

Again, with special reference to the preferred embodiment shown in the figures, the electrodes 8, 9 are arranged symmetrically with respect to the outlet hole 15. In even more detail, the main body 2 comprises, at the distal end 4, a housing seat 16 adapted to accommodate, at least partly, the tubular duct 10.

Preferably, the housing seat 16 has a substantially elongated and semi-cylindrical conformation.

Conveniently, the tubular duct 10 can be inserted within the housing seat 16 by shape coupling.

With special reference to the special embodiment shown in the figures, the housing seat 16 defines the outlet hole 15 at one relevant end.

According to the invention, the gripping portion 13 comprises coupling means 17 graspable by an operator or associable with at least one gripping device 18 of a robotic movement device 19 of the electrosurgical device 1.

In other words, while using the electrosurgical device 1, the user can grip it directly at the gripping portion 13 and move it.

Alternatively, the electrosurgical device 1 can be grasped by the gripping device 18 of the robotic movement device 19 which is controlled by the user in order to move the electrosurgical device 1.

Being able to combine the electrosurgical device 1 with the robotic movement device 19 allows the motility of the electrosurgical device 1 to be improved in terms of precision and accuracy, thus enabling the user to perform very precise surgical operations on the patient's body.

Conveniently, the coupling means 17 comprise at least one attachment body 20 made protruding cantilevered from the main body 2 and graspable by the gripping device 18 or by the operator.

Preferably, the attachment body 20 comprises:
at least one first attachment portion 21 substantially plate-shaped and associated with the main body 2;
at least one second attachment portion 22, substantially plate-shaped and substantially orthogonal to the first attachment portion 21.

The second attachment portion 22 is coupleable to the gripping device 18 or to the operator.

Advantageously, the main body 2 comprises at least one substantially flat main wall 23.

The first attachment portion 21 is associated with the main wall 23 in a protruding manner and is substantially orthogonal to the latter.

The presence of the main wall 23 promotes the grip of the attachment body 20 by the gripping device 18 or the user.

With particular reference to the preferred embodiment shown in the figures, the first attachment portion 21 and the second attachment portion 22 have a substantially trapezoidal cross-section.

In detail, the first attachment portion 21 and the second attachment portion 22 have a cross-section, drawn along a plane substantially parallel to the main wall 23, substantially trapezoidal and diverging toward the distal end 4.

This particular geometric conformation of the first attachment portion 21 and of the second attachment portion 22 promotes the grip of the attachment body 20 by the gripping device 18.

This will become clearer later, when the detail of the gripping device 18 is described.

Conveniently, the second attachment portion 22 comprises two extremal portions 24 protruding from the first attachment portion 21 and having a direction of longitudinal development diverging toward the distal end 4.

Next, the gripping device 18 will be described in detail, and it will become clear how the presence of the extremal portions 24 promotes the coupling between the gripping device 18 and the attachment body 20, thus preventing the accidental disconnection between these.

Again, as will be described later, the special geometric conformation of the extremal portions 24 promotes the placement of the gripping device 18 on the opposite side from the distal end 4, so as to interfere as little as possible with the placement of the electrodes 8, 9 on the patient's body.

Advantageously, the second attachment portion 22 has marginal edges 25 with a convex profile of curvilinear conformation.

This particular technical expedient further improves the grip of the attachment body 20 by the gripping device 18, as will become clear later in this disclosure. Conveniently, the main body 2 comprises at least one first shell element 26 and at least one second shell element 27.

The first shell element 26 and the second shell element 27 are associated along respective joining edges 28.

The coupling means 17 are associated with the first shell element 26.

The first shell element 26 comprises the main wall 23.

The first shell element 26 and the second shell element 27 extend longitudinally along the axis of longitudinal development of the main body 2.

Conveniently, the main body 2 comprises interlocking joining means 29 which are adapted to associate, through an interlocking coupling, the first shell element 26 with the second shell element 27.

With particular reference to the preferred embodiment shown in the figures, the interlocking joining means 29 comprise at least one protruding body 30 made on at least one of either the first shell element 26 or the second shell element 27, and a cavity (not visible in the figures) made on the other of either the first shell element 26 or the second shell element 27.

With particular reference to the preferred embodiment shown in the figures, the protruding body 30 is made on the second shell element 27 and the cavity is made on the first shell element 26.

The protruding body 30 can be inserted by interlocking into the cavity when the shell elements 26, 27 are mutually associated.

Conveniently, the protruding body 30 and cavity face toward the inside of the main body 2 when the shell elements 26, 27 are mutually associated.

Again, the interlocking joining means 29 comprise a pair of protruding elements 32, made on at least one of either the first shell element 26 or the second shell element 27, and a pair of recesses 33, made on the other of either the first shell element 26 or the second shell element 27.

The protruding elements 32 and the recesses 33 have a substantially elongated conformation along the axis of longitudinal development of the main body 2.

Conveniently, the protruding elements 32 and the recesses 33 extend substantially along the entire length of the main body 2.

With particular reference to the embodiment shown in the figures, the recesses 33 are made on the second shell element 27 and the protruding elements 32 are made on the first shell element 26.

Each of the protruding elements 32 is insertable by shape coupling into a respective recess 33, when the shell elements 26, 27 are mutually associated.

Making the protruding elements 32 and the recesses 33 substantially equal in length to the length of the main body 2 allows for improved interlocking between the first shell element 26 and the second shell element 27.

Again, the interlocking joining means 29 comprise a pair of securing bodies 34 made on the first shell element 26 and adapted to restrict the escape of the protruding elements 32 from their respective recesses 33.

Conveniently, the protruding elements 32, the recesses 33 and the securing bodies 34 face toward the inside of the main body 2 when the shell elements 26, 27 are mutually associated.

Preferably, the active electrode 8 and the neutral electrode 9 are arranged where the joining edges 28 of the first shell element 26 and of the second shell element 27 are located.

In detail, the main body 2 defines a pair of through holes partly defined by the joining edges 28 of the first shell element 26 and partly by the joining edges 28 of the second shell element 27.

The electrodes 8, 9 are adapted to escape from the distal end 4 each through the respective through hole defined by the joining edges 28.

With particular reference to the embodiment shown in the figures, the outlet hole 15 is defined by the joining edges 28 of the first shell element 26 and by the joining edges 28 of the second shell element 27.

Conveniently, the electrosurgical device 1 comprises at least one suction duct 35 of biological residues passing through the main body 2 and connectable to the suction means 36.

In detail, the suction duct 35 comprises at least one suction port 37 of biological residues, arranged at the distal end 4, and a draining port 38 of biological residues, arranged at the proximal end 3 and connectable to the suction means 36.

With particular reference to the preferred embodiment shown in the figures, the suction port 37 is made on the second shell element 27.

Again, with particular reference to the preferred embodiment shown in the figures, the suction duct 35 is made as a single body piece with the second shell element 27.

Alternative embodiments of the electrosurgical device 1 cannot be ruled out wherein the suction duct 35 may be separate from the second shell element 27 and be inserted within the main body 2.

Conveniently, the electrosurgical device 1 comprises a tubular connecting element 47 associated with the draining port 38 of the suction duct 35 in a fluid-operated manner and connectable to the suction means 36.

A further aspect of the present invention relates to a kit 39 for the electrosurgical treatment of a patient.

The kit 39 comprises:
at least one electrosurgical device 1 according to one or more of the previously described embodiments;
at least one current generator 5 associable with the electrosurgical device 1 and configured to supply electrical power to the latter;
feeding means 6 associable with the electrosurgical device 1 and configured to supply the latter with at least one electro-conductive fluid 7; and
at least one robotic movement device 19 provided with at least one gripping device 18 coupleable to the attachment body 20 of the electrosurgical device 1.

The robotic movement device 19 is configured to allow for the movement of the electrosurgical device 1.

The kit 39 comprises a supporting structure 40 arranged to rest on the ground by interposition of wheeled elements 41 adapted to allow the ground movement of the supporting structure 40.

The kit 39 comprises suction means 36 of the biological liquids connectable to the suction duct 35 of the electrosurgical device 1.

The kit 39 comprises a control unit 42 associated with the supporting structure 40 and comprising the current generator 5 and the suction means 36 and configured to manage the operation thereof.

The control unit 42 is operationally connected to the feeding means 6 and is configured to manage the operation thereof.

The kit 39 comprises connecting means 43 configured to connect the control unit 42 to the electrosurgical device 1.

Conveniently, the gripping device 18 comprises clamping means 44 adapted to grasp the second attachment portion 22.

In detail, the clamping means 44 comprise two branch elements 45 hinged together and provided with a through slot 46 adapted to accommodate the extremal portions 24.

Usefully, the through slots 46 have a complementary shape with the extremal portions 24.

If the robotic movement device 19 is used to move the electrosurgical device 1, the clamping means 44 are placed at the attachment body 20 and grasp it from the opposite side with respect to the distal end 4.

In this way, the gripping device 18 does not interfere with the electrodes 8, 9 during their placement at the patient's structures and/or organs.

The special technical expedient of making the first attachment portion 21 and the second attachment portion 22 with a substantially trapezoidal cross-section and diverging toward the distal end 4 promotes the gripping phase of the clamping means 44 at the attachment body 20.

Similarly, the fact that the second attachment portion 22 may comprise two extremal portions 24 protruding from the first attachment portion 21 and having a diverging direction of longitudinal development toward the distal end 4 promotes the grip of the branch elements 45 which go substantially parallel to the protruding ends 24.

This aspect increases the adhesion surface between the second attachment portion 22 and the clamping means 44, thus ensuring a stable coupling between the gripping device 18 and the attachment body 20.

Again, the presence of the through slots 46, being complementary in shape with the protruding ends 24, promotes the snug fitting of the latter within the through slots 46, helping to define a stable grip of the gripping device 18 on the attachment body 20.

It has in practice been ascertained that the described invention achieves the intended objects.

In particular, the fact that the gripping portion of the electrosurgical device according to the invention may be provided with an attachment body that can be grasped by a gripping device of a robotic movement device allows the electrosurgical device to be moved with improved precision compared to the case of manual handling by an operator.

In this way, the electrosurgical device according to the invention can be used during very delicate and fine surgical operations and/or to be performed in confined spaces.

1) Electrosurgical device (1), comprising:
at least one substantially hollow main body (2) of elongated conformation and provided with a proximal end (3), connectable to a current generator (5) and to feeding means (6) of an electro-conductive fluid (7), and with a distal end (4) provided with at least one active electrode (8) and with at least one neutral electrode (9) adapted to contact the body of a patient; and
at least one tubular duct (10) passing through said main body (2) and provided with at least one feeding hole (11) of said fluid (7) and with at least one dispensing hole (12) of said fluid (7);
a gripping portion (13) associated with said main body (2);
characterized by the fact that said gripping portion (13) comprises coupling means (17) graspable by an operator or associable with at least one gripping device (18) of a robotic movement device (19) of said electrosurgical device (1).

2) Electrosurgical device (1) according to claim 1, characterized by the fact that said coupling means (17) comprise at least one attachment body (20) made protruding cantilevered from said main body (2) and graspable by said gripping device (18) or by said operator.

3) Electrosurgical device (1) according to claim 1, characterized by the fact that said attachment body (20) comprises:
at least one first attachment portion (21) substantially plate-shaped and associated with said main body (2);
at least one second attachment portion (22), substantially plate-shaped and substantially orthogonal to said first attachment portion (21), said second attachment portion (22) being coupleable to said gripping device (18) or to said operator.

4) Electrosurgical device (1) according to claim 1, characterized by the fact that said main body (2) comprises at least one substantially flat main wall (23), said first attachment portion (21) being associated with said main wall (23) in a protruding manner and being substantially orthogonal to the latter.

5) Electrosurgical device (1) according to claim 1, characterized by the fact that said first attachment portion (21) and said second attachment portion (22) have a substantially trapezoidal cross-section.

6) Electrosurgical device (1) according to claim 1, characterized by the fact that said second attachment portion (22) comprises two extremal portions (24) protruding from said first attachment portion (21) and having a direction of longitudinal development diverging towards said distal end (4).

7) Electrosurgical device (1) according to claim 1, characterized by the fact that said second attachment portion (22) has marginal edges (25) with a convex profile of curvilinear conformation.

8) Electrosurgical device (1) according to claim 1, characterized by the fact that said main body (2) comprises at least one first shell element (26) and at least one second shell element (27), said first shell element (26) and said second shell element (27) being associated along respective joining edges (28), said coupling means (17) being associated with said first shell element (26).

9) Electrosurgical device (1) according to claim 1, characterized by the fact that said active electrode (8) and said neutral electrode (9) are arranged where said joining edges (28) of said first shell element (26) and of said second shell element (27) are located.

10) Kit (39) for the electrosurgical treatment of a patient, characterized by the fact that it comprises:
at least one electrosurgical device (1) according to claim 1;
at least one current generator (5) associable with said electrosurgical device (1) and configured to supply electrical power to the latter;
feeding means (6) associable with said electrosurgical device (1) and configured to supply the latter with at least one electro-conductive fluid (7); and
at least one robotic movement device (19) provided with at least one gripping device (18) coupleable to said attachment body (20) of said electrosurgical device (1), said robotic movement device (19) being configured to allow for the movement of said electrosurgical device (1).

11) Kit (39) according to claim 10, characterized by the fact that said gripping device (18) comprises clamping means (44) adapted to grasp said second attachment portion (22).

12) Kit (39) according to claim 10, characterized by the fact that said clamping means comprise two branch elements (45) hinged together and provided with a through slot (46) adapted to accommodate said extremal portions (24).

13) Kit (39) according to claim 10, characterized by the fact that said through slots (46) have a complementary shape with said extremal portions (24).
